(19) United States
(12) Patent Application Publication
(10) Pub. No.: US 2024/0350122 A1
(43) Pub. Date: Oct. 24, 2024

(54) DIAGNOSTIC IMAGING SYSTEM

(71) Applicant: RMI Oceania PTY LTD, VIC (AU)

(72) Inventors: Ajay Kumar Pandey, Brisbane Qld (AU); Davide Fontanarosa, St Kilda (AU); Maria Antico, St Kilda (AU); Mario Strydom, St Kilda (AU)

(21) Appl. No.: 18/686,172

(22) PCT Filed: Aug. 23, 2022
(86) PCT No.: PCT/AU2022/050957

§ 371 Date: Feb. 23, 2024

(30) Foreign Application Priority Data
Aug. 24, 2021 (AU) 2021902680

(51) Int. Cl. A61B 8/00

(52) U.S. Cl. CPC A61B 8/565; A61B 8/4218; A61B 8/4254; A61B 8/461

(57) ABSTRACT

A diagnostic imaging system comprising: a manipulator arm of a robot, the manipulator arm or robot system, comprising a plurality of elements interconnected to each other by a plurality of joints whereby each element is rotatable relative to an adjoining element of the manipulator arm; a diagnostic ultrasound probe module comprising an ultrasound transducer, the diagnostic module being coupled to one of the elements of the manipulator arm by a coupling arrangement to allow movement of the diagnostic module relative to said one of the elements of the manipulator arm; a controller coupled to the manipulator arm and coupling arrangement to employ the manipulator arm to move the diagnostic ultrasound probe module relative to a subject's anatomy, the subject being positioned on a bed frame, wherein the controller is arranged to control the movement of the plurality of elements and the coupling assembly in a plurality of operable modes such that in each operable mode, motion of the ultrasound probe module relative to the subject's anatomy is limited by limiting movement of one or more of the joints of the manipulator arm in said operable mode to limit motion of the diagnostic ultrasound probe module within a pre-defined range.

### TECHNICAL FIELD

The present disclosure relates to a diagnostic imaging system and a method of visualising diagnostic images by facilitating control of the diagnostic system from a remote location.

### BACKGROUND

Any references to methods, apparatus or documents of the prior art are not to be taken as constituting any evidence or admission that they formed, or form part of the common general knowledge.

Robotics started to solve medical challenges in the 1940s, but their utility was only realised in the early 1980s from industrial systems. Over the past few decades, robots have grown in precision and complexity for various medical applications. Robots have significant advantages in delivering more precise manoeuvres in the examination room, reducing unintended damage and shortened examination times for patients. The primary role of robots is to improve the safety, success, and consistency of medical procedures. Modern medical robots present in many forms, with applications ranging from complex joint replacements to rehabilitation. Most robotic systems have multiple integrated sensors to measure, track, align, and analyse the patient and environment. Some sensors are invasive and installed on or in patients, while others (e.g., electro-optical cameras) are non-invasive.

Medical support through robotics is developing rapidly due to the increasing demand for remote capabilities and alleviating the stresses on the operator. Progress has been made in autonomous surgical manoeuvres, optical coherence, tomography guidance, worldwide high-speed data connectivity and motion compensation, revolutionising remote surgical control applications. However, research has overlooked these technological advantages in the context of autonomous remote cardiac ultrasound imaging applications. To the best of our knowledge, there is currently no commercially implemented ultrasound imaging system which allows robots to autonomously perform the ultrasound imaging, using the same movements as sonographers on a patient, and interpret the scanned image to deliver a cardiac diagnosis.

Ultrasound scanning is a low-risk, low-impact medical procedure that, with full automation, could significantly reduce the cost to ultrasound scanning organisations and lower the impact on sonographers. Additionally, the synergy between medical and robotic technologies could improve access to cardiac ultrasound imaging to remote communities, which previously did not have such facilities-ultimately saving lives. It is desirable to provide a robotic system in combination with ultrasound diagnostic equipment to reduce the impact on sonographers locally, and support remote cardiac imaging operated by a remotely located sonographer that is comparable to an in-room scan.

### SUMMARY OF INVENTION

In an aspect, the invention provides a diagnostic imaging system comprising:
a manipulator arm of a robot, the manipulator arm comprising a plurality of elements interconnected to each other by a plurality of joints whereby each element is rotatable relative to an adjoining element of the manipulator arm;
a diagnostic ultrasound probe module comprising an ultrasound transducer, the diagnostic module being coupled to one of the elements of the manipulator arm by a coupling arrangement to allow movement of the diagnostic module relative to said one of the elements of the manipulator arm;
a controller coupled to the manipulator arm and a coupling arrangement to employ the manipulator arm to move the diagnostic ultrasound probe module relative to one of a plurality of working regions of a subject's anatomy based on a selection of one out of a plurality of operating modes displayed on a control panel configured to receive user input, each of said plurality of operating modes corresponding to said plurality of working regions of the subject's anatomy, the subject being positioned on a bed frame, wherein the controller is arranged to control the movement of the plurality of elements and the coupling assembly in each of said plurality of operable modes such that in each operable mode, motion of the ultrasound probe module relative to the subject's anatomy is limited by limiting movement of one or more of the joints of the manipulator arm in said operable mode to limit motion of the diagnostic ultrasound probe module within a pre-defined range and limit contact of the probe module within the working region on the patient's body that corresponds to the selected operating mode.

In an embodiment, selection of any one of the said plurality of modes positions the elements of the manipulator arm into a predetermined starting position corresponding to the selected mode before allowing movement of the one or more of the joints of the manipulator arm in selected mode, by using the controller, within the pre-defined range for the selected mode to limit contact of the probe module within the working region on the patient's body that corresponds to the selected operating mode.

In an embodiment, switching between a first and second operable mode, based on a selection on the control panel, results in the positioning of the elements of the manipulator arm into the predetermined starting position for the first operable mode before effecting further movement and positioning of the elements of the manipulator to the predetermined starting position for the second operable mode thereby preventing collision between the subject's anatomy and the diagnostic probe when switching between the first and second operable modes.

In an embodiment, selection of each operable mode corresponds to a respective set of spatial limits on movement of the joints of the manipulator during operation in the selected mode.

In an embodiment, the diagnostic imaging system further comprises a user interface being provided on said control panel, control panel being in communication with the controller, the user interface comprising a display device to display visual representation of the plurality of operable modes, the input interface being configured to receive input from the user to effect selection of one of the operable modes.

In an embodiment, the display device is configured to present a visual or audio representation of a force being applied by the ultrasound probe on the subject's anatomy during use in each operable mode.

In an embodiment, the system further comprises a sensor positioned relative to the one or more elements of the manipulator arm for sensing force applied by manipulator arm on the subject, based on controlling input received by control from a user during operation in a selected operable mode wherein the sensor is coupled with a force feedback module that communicates with the controller and the coupling arrangement to limit or effect movement of the elements of the manipulator and at least partially override the controlling input provided by the user when the force sensed by the sensor exceeds a preset threshold value.

In an embodiment, the controller further comprises a feedback system to allow a user to operate the manipulator arm using haptic, audio or visual feedback.

Preferably, for each operable mode, the feedback controller is operated with a corresponding set of operational parameters to apply a specific scale factor and direction control to the feedback controller for each operable mode.

In an embodiment, during operation in any of the operating modes, the spatial position of the ultrasound probe can apply a constant force on the subject's anatomy during use at a specific position of interest, or during probe motion where it is necessary to follow the body contours of the patient.

In another aspect, the diagnostic imaging system comprises an additional body manipulator arm, the body manipulator arm comprising a base that is movably attached to a bed frame to allow movement of the additional body manipulator arm along the length of the bed frame, the body manipulator further comprising a plurality of movable elements arranged to support and move a lifting member configured to move parts of the subject anatomy to provide space for the ultrasonic probe to be moved closer to a region of the subject's anatomy and contact the region during use.

In an embodiment, the plurality of elements are interconnected by a plurality of joints and wherein each element of the additional body manipulator is rotatable relative to an adjoining element of the additional body manipulator arm.

Preferably, the base of the additional body manipulator arm is arranged to slide along the length of the bed frame to allow the additional body manipulator to be positioned in a plurality of supporting locations.

In an embodiment, the manipulator arm is mounted on a robotic trolley with at least one degree of freedom and preferably at least two degrees of freedom to undertake movement of the manipulator arm towards and away from the patient

In an embodiment, the manipulator arm comprises at least six degrees of freedom for imparting movement to the ultrasound probe module.

In an embodiment, the diagnostic imaging system further comprises a camera mounted at or adjacent the diagnostic ultrasound probe module on the robot arm to display location of the ultrasonic transducer relative to the subject's anatomy during use.

In another embodiment, the diagnostic imaging system further comprises a proximity sensor to sense distance between the diagnostic probe and the patient and wherein the proximity sensor is coupled with a proximity feedback module that communicates with the controller and the coupling arrangement to limit or effect movement of the elements of the manipulator and at least partially override the controlling input provided by the user when the distance sensed by the sensor exceeds one or more preset threshold values.

In another aspect, the invention provides method of visualising a diagnostic image, the method comprising:
positioning a manipulator arm of a robot at a proximate location relative to a subject positioned on a bed frame, the manipulator arm comprising a plurality of elements interconnected to each other by a plurality of joints whereby each element is rotatable relative to an adjoining element of the manipulator arm and wherein a diagnostic ultrasound probe module comprising an ultrasound transducer is coupled to one of the elements of the manipulator arm by a coupling arrangement to allow movement of the diagnostic module relative to said one of the elements of the manipulator arm;
controlling movement of the manipulator via a controller in communication with the manipulator arm and an operator interface, by selecting one out of a plurality of operating modes displayed on the operator interface configured to receive user input, each of said plurality of operating modes corresponding to said plurality of working regions of the subject's anatomy operating mode working region of the subject's anatomy-control panel, with a coupling arrangement to effect movement of the diagnostic ultrasound probe module relative to the subject's anatomy to limit movement of the ultrasonic probe module and the elements of the manipulator arm during operation in said the selected operable mode in a pre-defined range and limit contact of the probe module within the working region on the patient's body that corresponds to the selected operating mode.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is an isolated perspective of a primary manipulator arm 100 that forms a part of a diagnostic imaging system 1000 in accordance with an embodiment.

FIG. 2 depicts the primary manipulator arm 100 being used in combination with an optional secondary manipulator arm 600 which also forms part of the diagnostic imaging system 1000.

FIG. 3 depicts a visual display device 900 with pictographical representations of a plurality of operational modes for the diagnostic imaging system 1000 which include 8 operation modes (scanning windows) i.e., in terms of 2 parasternal, 4 apical, 2 subcostal.

FIG. 4 is pictographical display 400 of force being applied by the diagnostic ultrasound probe module on the subject's anatomy.

FIG. 5 is a box diagram of the diagnostic imaging system 1000.

FIG. 5A is a simplified box diagram of an embodiment of the imaging system 1000.

FIG. 6 is a flowchart of a method of conducting ultrasound cardiac imaging by utilising the diagnostic imaging system 1000.

FIG. 6 is a detailed flow diagram of an embodiment the diagnostic imaging system 1000 which includes an imaging module 500, a sonographer module 700 and a robotic module 300 (comprising the primary manipulator 100, the secondary manipulator 600 and the controller 350).

### DETAILED DESCRIPTION OF PREFERRED EMBODIMENTS

In a broad aspect, the invention provides a mobile, portable or fixed cardiac ultrasound imaging system 1000 which includes one or more robotic manipulator arms (as will be discussed in detail in the foregoing sections) for facilitating remote control of an ultrasound imaging system. Whilst, the present examples are limited to conducting cardiac ultrasound imaging, the scope of the invention is in no way limited to cardiac imaging only and could therefore be applied for ultrasound scanning and imaging of other anatomic parts of a subject.

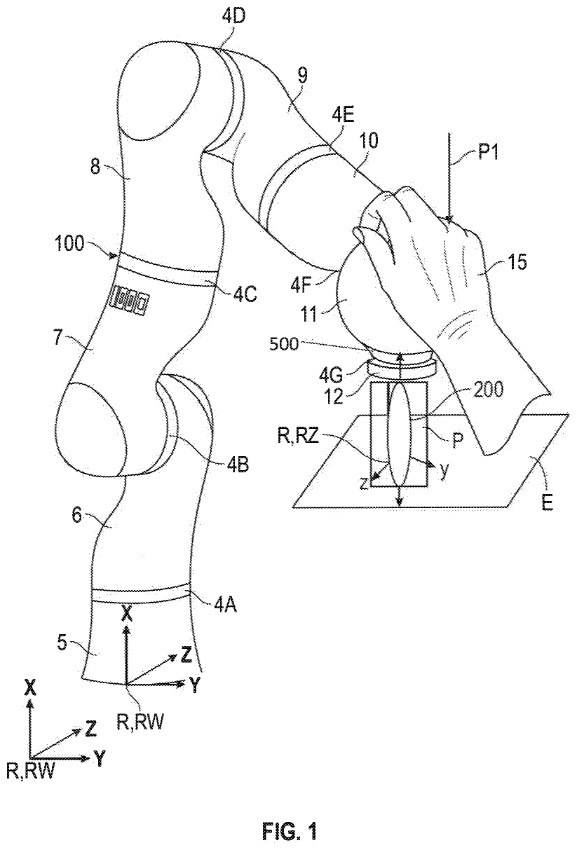

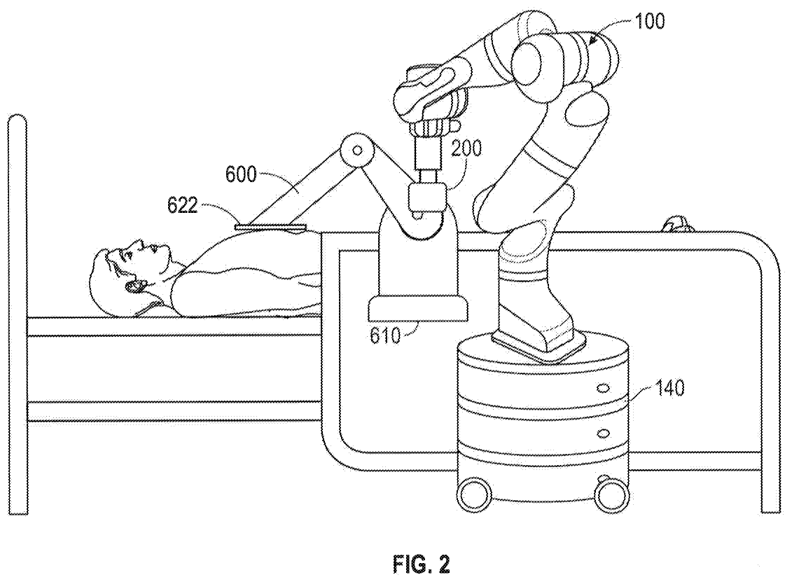

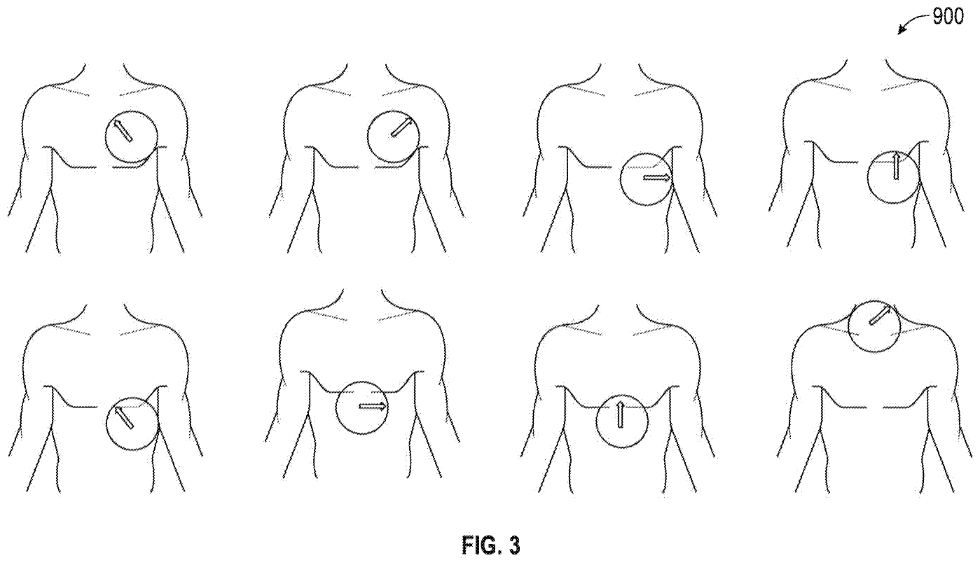

Referring to FIGS. 1 and 2 in particular, the diagnostic imaging system 1000 comprises a manipulator arm 100 such as a collaborative robot. In the presently described embodiment, the manipulator arm 100 comprises a plurality of movable elements 5, 6, 7, 8, 9, 10 and 11 that are interconnected to each other by joints 4A to 4F to effect rotational movement between adjacent elements. Element 5 may be positioned on a base (as shown in FIG. 2) which could either be fixed or be movably positioned on a trolley 140.

The robotic manipulator 100 comprises a fixed base with several movable parts that can move along a plurality of axes, each of the axes being located at the joints 4A to 4F with the each of the elements 5 to 11 with a diagnostic ultrasonic probe 20 being attached to lever 11. For the purposes of simplicity, each of the axes at joints 4A to 4F will be depicted by axes 1-6 in the written description. Each of the axes 1-6 is moved with a drive, for example an electric drive, which are electrically connected in a non-depicted manner to a controller 300 which includes a computer 310, so that the controller control computer 310 or a computer program running on control computer 310 is able to activate electric drives in the joints 4A to 4F in such a way that the position of element 12 to which the ultrasonic diagnostic device module 200 can be oriented essentially freely in space. The electric drives of the manipulator arm 100 each include for example an electric motor and possibly power electronics that activate the motors.

In the case of the present embodiment, these are reference coordinate systems R in the form of three-dimensional Cartesian coordinate systems each having six freedoms, particularly a world coordinate system RW of a workspace of the manipulator 100, a basic coordinate system RB in an element 5 of the manipulator 100 which forms a foot of the manipulator 100, or an ultrasonic diagnostic coordinate system RZ of the diagnostic module 200 attached to element 12 of the manipulator 100.

Movement between the plurality of elements along joints 4A to 4F is effected about each of the axes 1-6 with a drive, preferably electric drives so that the controller 300 is able to activate the electric drives in such a way so as to position or orient the ultrasonic diagnostic module 200 or its centre point freely in space.

In the case of the present exemplary embodiment, control computer 310 is programmed or designed in such a way that it, or a computer program running on it, is able to limit a work region A of the primary manipulator 100 corresponding to different operable modes. In FIG. 3, eight different cardiac ultrasound modes have been shown on a display device 900 presented to an operator. The display device 900 forms part of a control panel that is in electronic communication with the control computer 310. Each image in FIG. 3 depicts a specific operational mode and each operational mode not only corresponds to a specific working region but also orients the ultrasonic diagnostic module 200 in a specific orientation. Each work region corresponding to an operational mode is understood to mean the permissible zone for the manipulator robot 100 for working and traveling. During operation of the primary manipulator robot 100 in one of the eight operational modes, the work region and orientation for the ultrasonic diagnostic module 200 is spatially bound thereby defining a cardiac window for scanning. One of the key advantages provided by using pre-defined working regions for the manipulator robot 100 corresponding to a cardiac window is that movement within each window is limited to ensure safe operation of the primary manipulator robot 100. Once a cardiac window is selected by a user on the user input interface displayed on the display device 900, the controller 300 activates one of the plurality of activation modes, as selected by the user, and the manipulator arm 100 positions the ultrasonic diagnostic probe module 200 to a preset starting location that is unique for the selected operation mode. Specifically, selection of any one of the plurality of operation modes by the user positions the elements of the manipulator arm 100 into a predetermined starling position which is unique to the specific chosen operation mode. If another operation mode was chosen by the user, then the elements of the manipulator arm 100 would move to another starting position that corresponds to the other chosen operation mode. It is important to note that for each operable mode, the unique starting position coordinates for each operable mode may be saved on a memory device that is in communication or a part of the controller computer 310.

Choosing a specific operation limits allowable movement of the diagnostic probe module 200 within the corresponding working region that corresponds to the chosen operation mode and only allows certain pre-defined movements of the ultrasonic diagnostic module 200 thereby making operation of the primary manipulator 100 safer and preventing any inadvertent movements which might accidentally hurt the subject and also enables better image capture. Furthermore, switching between two operation modes, specifically a first and second operable mode, based on a selection on the control panel, results in the positioning of the elements of the manipulator arm 100 into the predetermined starting position for the first operable mode before effecting further movement and positioning of the elements of the manipulator to the predetermined starting position for the second operable mode thereby preventing collision between the subject's anatomy and the diagnostic probe when switching between the first and second operable modes. In many instances, the end user may commence movement of the manipulator arm 100 in the first mode to access various anatomical parts of the subject's body within the working region that corresponds to the first mode which implies that the diagnostic module 200 and the manipulator arm 100 may be positioned into one of many possible positions within the working zone that no longer corresponds to the initial predetermined starting position for the first mode. When the end user, switches from the first mode to the second mode, the controller automatically returns the manipulator arm 100 to the predetermined starting position for the first mode from the one of many possible locations within the first working zone. The system 100 prevents movement of the manipulator directly from any position within the first working region straight to the predetermined starting position of the second operable mode (which may inadvertently cause accident or hurt the subject) and forces the manipulator arm 100 to return to the predetermined starting position of the first mode before effecting further movement to the predetermined starting position of the second mode. As a result, any movements of the manipulator arm 100 and the diagnostic probe module 200 over larger ranges, in between operable modes are only carried out over safe switching paths between the operation modes which further enhances safety of the aforementioned diagnostic system 100.

During use, a subject or patient may lie on a bed frame (See FIG. 2) and the manipulator robot 100 may be manually or autonomously guided to a location close to the bed frame. This location may be a fixed reference location. The initial placement location for the primary manipulator robot 100 may vary depending on the physical characteristics of the subject. A dynamic placement location for the primary manipulator robot 100 may be computed based upon one or more markers positioned on the patient's anatomy. In this regard, the base of the primary manipulator robot 100 may be positioned on a robotic trolley 140 (shown in FIG. 2) with two degrees of freedom to allow movement and initial positioning of the manipulator robot 100.

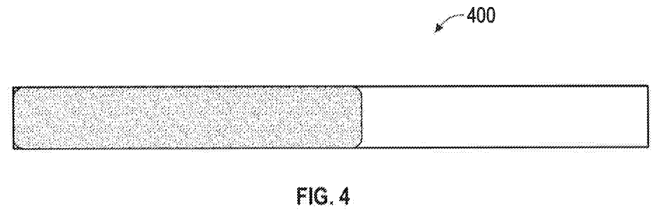

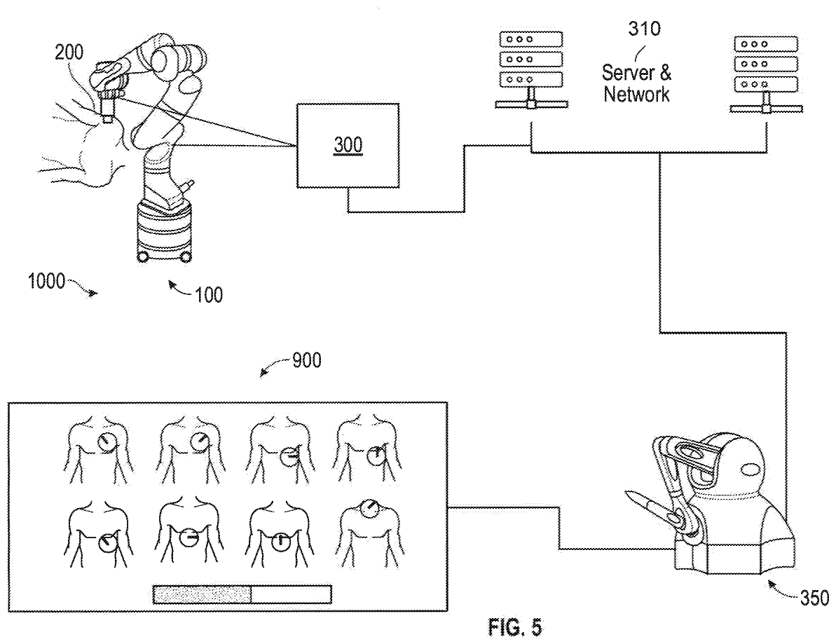

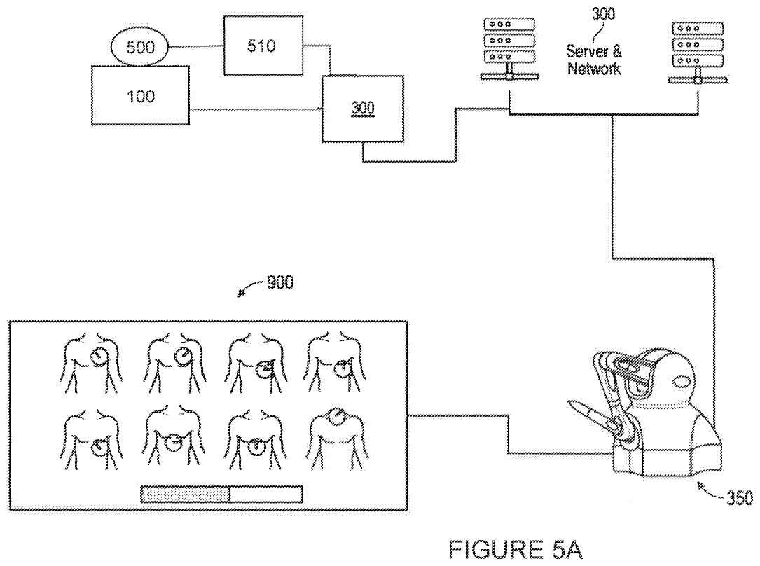

Echocardiograms may require pressing quite firmly or manipulating the ultrasound probe between ribs on the patient to obtain clear images, which mainly depends on the patient's anatomy and subcutaneous fat mass in the scanning window. The ultrasound robot pressure on the body (force) may therefore be varied depending on the anatomy of the patient. Moreover, the section of the operation window or scanning window may also limit the extent of force being applied by the manipulator robot 100. By way of example only, the force being applied by the manipulator arm 100 holding the ultrasonic diagnostic module 200 may change from 500 grams to 3 kg or more based on the operational mode being selected. Advantageously, as shown in FIG. 4, a scale 400 may be displayed on a display device viewable by an operator operating and controlling the manipulator robot 100 via the controller 300. Alternatively, an audible signal may also be used a cue to indicate the force being applied by the manipulator arm 100. For example, a higher pitch could represent a greater force. In a preferred embodiment, shown in FIG. 5A and FIG. 1), a force torque sensor 500 (such as, but not limited to, the FT 300-S Force Torque sensor from Robotiq) may be positioned on the diagnostic module 200 or relative to any one of the elements of the manipulator arm 100 to measure actual force or torque applied by the diagnostic module 200 on the patient's body based on controlling input received by control from a user during operation in a selected operable mode. The sensor 500 is coupled with a force feedback module 510 that communicates with the controller 300 and the coupling arrangement to limit or effect movement of the elements of the manipulator arm 100 and at least partially, or in some instances completely, override the controlling input provided by the user when the force sensed by the sensor 500 exceeds a preset threshold value. This is yet another important safety feature provided by the diagnostic imaging system 100 which further presents a viable way of carrying out remote ultrasound scanning of a patient without compromising on patient safety. In some alternative embodiments, the force torque sensor may be integrated into one or more elements that form the manipulator arm 100. In one embodiment, in each operable mode, the manipulator arm 100 limitation of movement may be further restrained or controlled by only allowing the user to control movement in five dimensions instead of six dimensions. During such a force controlled operation, the arm 100 may move semi-autonomously towards the patient's body and apply a pre-set level of force on the patient's body region associated with the selected operable mode.

In addition to the force torque sensor, a proximity sensor (which may be in the form of a camera mounted at or adjacent the ultrasonic diagnostic module 200). The proximity sensor senses distance between the diagnostic probe and the patient. The proximity sensor is coupled with a proximity feedback module that communicates with the controller and the coupling arrangement to limit or effect movement of the elements of the manipulator arm 100 and at least partially override the controlling input provided by the user when the distance sensed by the sensor exceeds one or more preset threshold values. The use of the proximity sensor enables automatic changing of velocity of movement of the manipulator arm 100. As the diagnostic module gets closer to the patient's body, the approach velocity of the manipulator arm 100 may slow down in response to the increasing proximity of the diagnostic module relative to the patient's body.

Each of the predetermined starting positions for the respective operating modes may be stored in a location array that may be saved in the memory device in communication with or which is a part of the control computer 310. The stored locations may include include Cartesian coordinates (e.g., XYZ coordinates) identifying the position the various elements of the manipulator arm 100 in three dimensions. For example, the location array saved on the memory device may include the position of the particular elements which form part of the manipulator arm 100. The pre-defined range of movement for the elements comprising the manipulator arm 100 is each operable mode is typically defined by a range of allowed movements of the elements (relative to each other) and locations or coordinates within which movement of the manipulator arm 100 would be permitted when each operable mode is selected by the user. It is important to note that in conventional manipulator arms, the elements forming the manipulator arms may typically move in a plurality of different ways to position the diagnostic probe at one location in a number of different orientations. The provision of only a fixed number allowable movements for the elements of the manipulator arm 100 in combination with limiting the working zone or area for the movement of the manipulator arm 100 not only enhances safety but also aids the remotely positioned controller in avoiding incorrect orientation of the diagnostic tool avoid any undesirable or unwanted orientations when conducting an ultrasound of important regions of the patient's body such as the heart.

FIG. 1 shows a user's hand 15 applying a force in direction P1. This action is nothing more than initialising the manipulator 100 to position the manipulator in a position that is at a safe distance away from the patient lying on a patient before handing control to a remotely located sonographer operator.

We refer to FIG. 5 which depicts a box diagram for the diagnostic system 1000. As previously discussed, the controller 300 may comprise a computer 310 or a processor in communication with the electrical motors of the manipulator robot 100 to effect relative movement of the plurality of elements 5, 6, 7, 8, 9, 10 and 11.

In the preferred embodiment, the system also includes a user control in communication with the controller 300 over a communication network and the user control may utilise haptic feedback. In other embodiments, a controller without haptic feedback may also be used. Specifically control of the manipulator robot 100 may be achieved by using haptic feedback received on a haptic feedback controller 350. In at least some embodiments, the manipulator robot 100 may include force sensors for sensing reactive force experienced by the manipulator robot 100 during movement of the ultrasonic diagnostic module 200 as it contacts the subject's anatomy whilst operation in one of the plurality of modes. Force is a measure within each robot joint (4A to 4F), from which the end-effector force can calculated, or directly measure on the end-effector. Using the pressure (robot force) exerted by the patient body on the probe, the pressure values may be fed back to the haptic controller 350 or audio feedback device (not shown), and the operator will effectively ‘feel’ the force on the patient's body through the force (or pressure) that creates a motion in that specific direction on the haptic controller 350. The pressure experienced by the patient is displayed on a bar graph on the display device, showing the sonographer the pressure on the patient. In order to further assist the operator controlling the haptic feedback controller 350, a camera may also be mounted at or adjacent the ultrasonic diagnostic module 200 to view images of the probe as it contacts the subject's anatomy. The visual and haptic feedback combines to provide the sonographer with an indication of the pressure or torque exerted on the patient during an ultrasound scan.

The end-effector is controlled by velocity rather than position to ensure safety and accuracy. The velocity of the manipulator robot's end affecter element 12 (which is attached to the ultrasonic diagnostic module 200) is controlled by the distance the haptic controller 350 is moved. For each scanning window (corresponding a specific operational mode), a different scale factor and direction control are calibrated into the haptic feedback controller 350 to ensure safe and effective manipulator robot motion at that position. In practice, if the user moves the haptic controller 350 further, the speed at which the manipulator robot's elements move becomes faster. An operator can move the controller 350, for instance, 10 mm and hold it there, and the robot will move at a fixed velocity until the controller position is changed. A desired position of the manipulator robot 100 may also be locked to hold the ultrasound diagnostic probe module 200 at a fixed position and apply a constant force on the anatomy of the subject (a feature which is almost impossible to achieve by a sonographer manually handling the ultrasonic probe). By making a slight movement with the haptic feedback controller 350, the robot motion is nearly invisible to a human. It implies the sonographer can accurately pin-point an anatomical position by controlling the robot speed and end-effector position to millimetre accuracy.

Using the haptic controller 350, the operator can either translate or rotate the probe that forms a part of the ultrasonic diagnostic module 200. Translation speed depends on the distance the controller 350 is moved. Rotation of the probe—or ‘rocking the probe’, changes the probe angle relative to the scanning position, which is essential for changing the view window of the heart. A significant advantage of using the manipulator robot 100 to scan instead of a sonographer's hand is that the manipulator robot 100 can precisely hold the position and pressure at that point—for as long as the operator needs to capture images or do adjustments. Any movements by the patient are followed by the robot to retain the force and position on the patient.

One or more additional cameras may also be provided to allow a remotely located sonographer to view and possibly communicate with the patient relative to the manipulator arm 100. The one or more additional cameras may communicate with the controller to display captured images on a display screen being viewed by the remotely located sonographer.

The diagnostic imaging system 1000 may also include a secondary manipulator 600 shown in FIG. 2. The secondary manipulator robot 600 which includes a movably disposed base 610 to allow sliding movement of the secondary manipulator robot 600 along the length of the bed frame upon which the subject is lying. The secondary manipulator arm 600 also includes a plurality of elements connected by rotatable joints with an effector end part that includes a lifting member in the form of a lifting plate 622. The lifting plate 622 is provided to allow parts of the subject's anatomy such as excess fat or anatomical body parts such as breasts to be moved out of the way, to provide space for the ultrasonic probe to be moved closer to a region of the subject's anatomy and effectively contact the region during use. The secondary manipulator 600 may be optionally utilised in one or more of the operational modes of the diagnostic imaging system 1000. Specifically, in some instances, the anatomical region to be scanned may not be readily accessible by the manipulator robot 100. In such instances, the secondary bed frame mounted manipulator arm 600 provides sufficient room to allow the manipulator robot 100 to be pre-positioned in accordance one of said operational modes to carry out scanning in the corresponding scanning window.

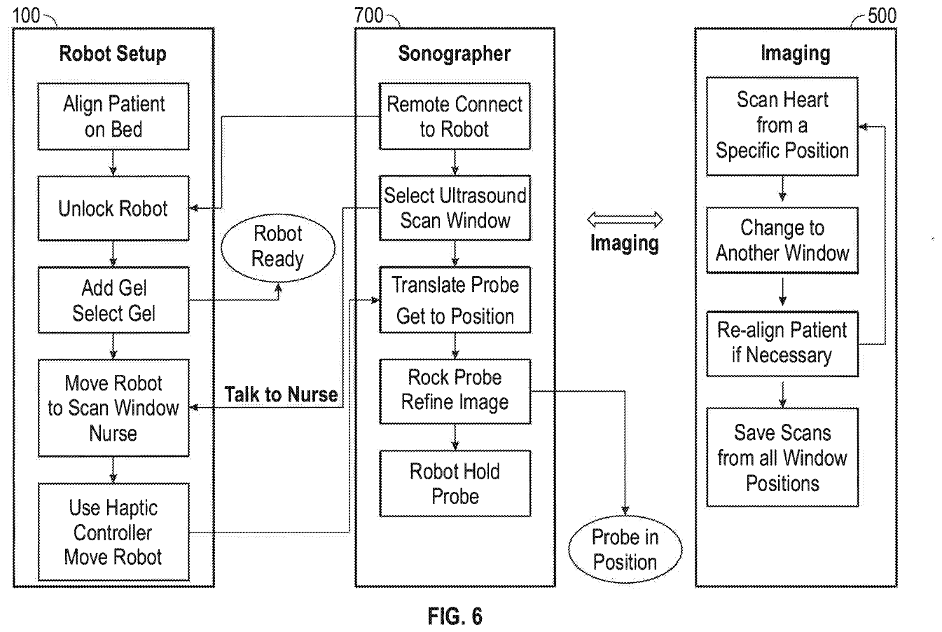

The use of the diagnostic imaging system 1000 utilizes highly specialized robotic manipulators 100 and 600 in combination with an ultrasound module and a specifically programmed controller module 300 to enable improved ultrasound scanning. Typical use of the system 1000 would involve a nurse in the room directing the subject to the bed frame in a manual teleoperation mode. The sonographer would use the controller 300 to align the primary robotic manipulator 100 by activating one of the operational modes via the user input interface which would involve selection of one of the cardiac windows displayed on the display device 900. In some instances, the nurse may need to move the base of the manipulator 100 to position the manipulator robot 100 in close proximity to the bed frame before selecting one of the operational modes to position the ultrasound probe 200 and perform a scan within a scanning window corresponding to the operational mode. Once the manual robot 100's robot end-effector is positioned on one of the acoustic windows, the robotic arm's motion is constrained to a defined, limited space. A key advantage of the robot is it can hold the probe for an extended period in one position. The spatial limits are defined based on the dimension of the area scanned during a regular echocardiogram. Thus, this solution will not affect the scanning procedure. Still, it will slow the probe velocity down and prevent the sonographer from performing inadvertent movements outside of the scanning window selected, that might result in a possible collision between the robot and the patient. The sonographers may further define one or more body landmarks to further confining the scanning region for each acoustic window. A detailed flow diagram of this process has been shown in FIG. 6.

In a typical operation, the sonographer may be located at a remote location and may connect to the robotic manipulators 100 and possibly 600 via the controller module 300 over a local LAN or WAN connection, unlock the robotic manipulators 100 and possibly 600 and request ask the nurse located in close proximity to the patient to align the patient. Once the patient has been aligned at a starting location, a scan window may be selected on the user input interface to move the primary manipulator 100 and specifically the ultrasonic module 200 mounted on the robot end effector towards the subject's anatomy. From this point, the remotely located sonographer can perform ultrasound scanning using the controller module 300. With the diagnostic probe 200 in position, the sonographer may translate the desired anatomical window (such as the heart during cardiac imaging) to find the optimum image. Once a good image is obtained, the sonographer can rock the probe by using the haptic feedback controller 350 accurately at that position to fine-tune the image.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features or ultrasound scanning windows shown or described since the means herein described comprises preferred forms of putting the invention into effect.

The invention is, therefore, claimed in any of its forms or modifications within the proper scope of the appended claims appropriately interpreted by those skilled in the art.

1. A diagnostic imaging system comprising:
a manipulator arm of a robot, the manipulator arm comprising a plurality of elements interconnected to each other by a plurality of joints whereby each element is rotatable relative to an adjoining element of the manipulator arm;
a diagnostic ultrasound probe module comprising an ultrasound transducer, the diagnostic module being coupled to one of the elements of the manipulator arm by a coupling arrangement to allow movement of the diagnostic module relative to said one of the elements of the manipulator arm;
a controller coupled to the manipulator arm and the coupling arrangement to employ the manipulator arm to move the diagnostic ultrasound probe module relative to one of a plurality of working regions of a subject's anatomy based on a selection of one out of a plurality of operating modes displayed on a control panel configured to receive user input, each of said plurality of operating modes corresponding to a respective one of said plurality of working regions of the subject's anatomy, the subject being positioned on a bed frame, wherein the controller is arranged to control the movement of the plurality of elements and the coupling assembly in each of said plurality of operable modes such that in each operable mode, motion of the ultrasound probe module relative to the subject's anatomy is limited by limiting movement of one or more of the joints of the manipulator arm in said operating mode to limit motion of the diagnostic ultrasound probe module within a pre-defined range and limit contact of the probe module within the working region of the subject's anatomy that corresponds to the selected operating mode.

2. A diagnostic imaging system in accordance with claim 1 wherein selection of any one of the said plurality of operating modes positions the elements of the manipulator arm into a predetermined starting position corresponding to the selected mode before allowing movement of the one or more of the joints of the manipulator arm in selected mode, by using the controller, within the pre-defined range for the selected mode to limit contact of the probe module within the working region on the patient's body that corresponds to the selected operating mode.

3. A diagnostic imaging system in accordance with claim 2 wherein switching between a first and second operable mode, based on a selection on the control panel, results in the positioning of the elements of the manipulator arm into the predetermined starting position for the first operable mode before effecting further movement and positioning of the elements of the manipulator arm to the predetermined starting position for the second operable mode thereby preventing collision between the subject's anatomy and the diagnostic ultrasonic probe module when switching between the first and second operating modes.

4. A diagnostic imaging system in accordance with claim 1 wherein selection of each operating mode corresponds to a respective set of spatial limits on movement of the joints and the diagnostic ultrasonic probe module of the manipulator arm during operation in the selected mode.

5. A diagnostic imaging system in accordance with claim 1 further comprising a user interface on said control panel in communication with the controller, the user interface comprising a display device to display visual representation of the plurality of operating modes, the input interface being configured to receive input from the user to effect selection of one of the operating modes.

6. A diagnostic imaging system in accordance with claim 5 wherein the display device is configured to present a visual or audible representation of a force being applied by the ultrasound probe on the subject's anatomy during use in each operating mode.

7. A diagnostic imaging system in accordance with claim 1 further comprising a sensor positioned relative to the one or more elements of the manipulator arm for sensing force applied by manipulator arm on the subject, based on controlling input received by control from a user during operation in a selected operating mode wherein the sensor is coupled with a force feedback module that communicates with the controller and the coupling arrangement to limit or effect movement of the elements of the manipulator arm and at least partially override the controlling input provided by the user when the force sensed by the sensor exceeds a preset threshold value.

8. A diagnostic imaging system in accordance with claim 1 wherein the controller further comprises a feedback controller to allow a user to operate the manipulator arm using haptic feedback.

9. A diagnostic imaging system in accordance with claim 1 wherein for each operating mode, the haptic feedback controller is operated with a corresponding set of operational parameters to apply a specific scale factor and direction control to the feedback controller for each operable mode.

10. A diagnostic imaging system in accordance with claim 1 wherein during operation in any of the operating modes, the spatial position of the ultrasound probe is operable to be temporarily locked to apply and set a constant force on the subject's anatomy during use.

11. A diagnostic imaging system in accordance with claim 1 further comprising an additional body manipulator arm, the body manipulator arm comprising a base that is movably attached to the bed frame to allow movement of the additional body manipulator arm along the length of the bed frame, the body manipulator further comprising a plurality of movable elements arranged to support and move a lifting member configured to move parts of the subject anatomy to provide space for the ultrasonic probe to be moved closer to a region of the subject's anatomy and contact the region during use.

12. A diagnostic imaging system in accordance with claim 11 wherein the plurality of movable elements are interconnected by a plurality of joints and wherein each element of the additional body manipulator arm is rotatable relative to an adjoining element of the additional body manipulator arm.

13. A diagnostic imaging system in accordance with claim 11 wherein the base of the additional body manipulator arm is arranged to slide along or across the length of the bed frame to allow the additional body manipulator arm to be positioned in a plurality of supporting locations.

14. A diagnostic imaging system in accordance with claim 1 wherein the primary manipulator arm is mounted on a robotic trolley or the medical bed, with at least one degree of freedom and preferably at least two degrees of freedom to undertake movement of the primary manipulator arm towards and away from the subject.

15. A diagnostic imaging system in accordance with claim 1 wherein the primary manipulator arm comprises at least six degrees of freedom for imparting movement to the ultrasound probe module.

16. A diagnostic imaging system in accordance with claim 1 further comprising a camera mounted at or adjacent the diagnostic ultrasound probe module to display location of the diagnostic ultrasonic probe module relative to the subject's anatomy during use.

17. A diagnostic imaging system in accordance with claim 1 further comprising a proximity sensor to sense distance between the diagnostic ultrasonic probe module and the subject and wherein the proximity sensor is coupled with a proximity feedback module that communicates with the controller and the coupling arrangement to limit or effect movement of the elements of the manipulator arm and at least partially override the controlling input provided by the user when the distance sensed by the sensor exceeds one or more preset threshold values.

18. A method of visualising a diagnostic image, the method comprising:
positioning a manipulator arm of a robot at a proximate location relative to a subject positioned on a bed frame, the manipulator arm comprising a plurality of elements interconnected to each other by a plurality of joints whereby each element is rotatable relative to an adjoining element of the manipulator arm and wherein a diagnostic ultrasound probe module comprising an ultrasound transducer is coupled to one of the elements of the manipulator arm by a coupling arrangement to allow movement of the diagnostic ultrasonic probe module relative to said one of the elements of the manipulator arm;
controlling movement of the manipulator via a controller in communication with the manipulator arm and an operator interface, by selecting one out of a plurality of operating modes displayed on the operator interface, the operator interface being configured to receive user input, each of said plurality of operating modes corresponding to a respective one of a plurality of working regions of the subject's anatomy, with the coupling arrangement to effect movement of the diagnostic ultrasound probe module relative to the subject's anatomy to limit movement of the diagnostic ultrasonic probe module and the elements of the manipulator arm during operation in said the selected operating mode in a pre-defined range and limit contact of the probe module within the working region of the subject's anatomy that corresponds to the selected operating mode.
